(19) United States
(12) Patent Application Publication
(10) Pub. No.: US 2006/0004191 A1
(43) Pub. Date: Jan. 5, 2006

(54) Modified sodium iodide symporter proteins and genes for imaging and cancer therapy

(76) Inventors: Sissy M Jhiang, Columbus, OH (US); Daniel Hy Shen, Columbus, OH (US); Xiaoqin Lin, Columbus, OH (US)

Correspondence Address: Sissy M Jhiang, 1492 Marland Street, Columbus, OH, 43238, US

(21) Appl. No.: 10/519,183

(22) PCT Filed: Jun. 25, 2003
(86) PCT No.: PCT/US03/20111

§ 371 Date: Dec. 21, 2004

Related U.S. Application Data
(60) Provisional application No. 60/391,285, filed on Jun. 25, 2002.

(30) Foreign Application Priority Data
Jun. 25, 2002 (US) 60/391,285

(51) Int. Cl. C12Q 1/68; C07H 21/04; C07K 14/705

(52) U.S. Cl. 536/23.5; 530/350; 435/6; 435/69.1; 435/320.1; 435/325

(57) ABSTRACT

The invention provides modified sodium iodide symporter (NIS) proteins, and polynucleotides encoding modified NIS proteins. The modified NIS proteins have a net electrostatic charge more positive than that of corresponding wild-type NIS proteins. Expression of a modified NIS protein in a cell results in higher intracellular concentrations of NIS substrates as compared to equivalent expression of the corresponding wild-type NIS protein in a cell. The invention also provides methods for imaging cells or for therapy of cancer cells, using the modified NIS proteins to facilitate transport of NIS substrates into cells in the body of an individual. Uptake of NIS substrates suitable for either imaging or that have cytotoxic activity, into the cells of the individual that express modified NIS protein facilitate detection of the substrate (for imaging) or killing of the cancer cells (for therapy).

This application claims priority from U.S. provisional patent application No. 60/391,285, filed Jun. 25, 2002, which is herein incorporated by reference.

The work described in this application was supported, at least in part, by Grant No. RO1 CA60074 from the National Institutes of Health. The U.S. Government has certain rights in this invention.

### FIELD OF INVENTION

The invention relates to modified sodium iodide symporter (NIS) proteins, whose expression increases the intracellular concentration of NIS substrates, and polynucleotides encoding modified NIS proteins, The invention also relates to methods for increasing the concentration of NIS substrates in cells, particularly cancer cells, of an animal for the purposes of scintigraphic imaging or therapy.

### BACKGROUND

Iodine is an essential component of thyroid hormones. Because thyroid hormone is made in the thyroid gland and because iodine is a rare element, the thyroid gland of animals has an effective method of sequestering iodide, the anionic form of iodine, from the blood circulation for use in the synthesis of thyroid hormones. This method of sequestering iodide is dependent on expression of the sodium iodide symporter (Na⁺/I⁻ symporter or NIS) gene and protein within cells of the thyroid gland. Expression of this symporter results in uptake of iodide across the basolateral membrane of thyroid follicular cells in an active transport process. NIS protein is an intrinsic membrane protein with 13 putative transmembrane domains^{1,2,25}. NIS protein transports one I⁻ ion with two Na⁺ ions into cells²⁶. NIS expressing-tissues also concentrate pertechnate (TcO₄⁻)²⁷, perrhenate (ReO₄⁻)^{27,28}, and astatide (At⁻)^{29,30}. Such molecules that are transported by NIS proteins are said to be substrates of NIS proteins or NIS substrates. NIS is expressed primarily in thyroid tissues, but variable degrees of NIS expression are present in various other tissues including nasal mucosa, stomach, salivary glands, and lactating breast tissues. NIS transport of iodide is competitively inhibited by the anions thiocyanate and perchlorate.

Radioiodide concentrating activity in the thyroid has provided methods for treating patients with thyroid cancers and for imaging such cancers. Radioiodide concentrating activity can also be used to diagnose decreased ability of the thyroid to take up iodide. In these methods, thyroid cells take up iodide, preferably radioactive isotopic iodide. The effectiveness of such treatment and imaging, however, is hampered due to the low activity of NIS protein in the cells. Such low NIS levels are at least partially brought about by decreased expression of NIS in some thyroid tumors as compared to non-tumor cells. The short time that such radioiodide is retained inside the cell is also limiting. In addition, the treatment and imaging methods are useful only for cells and tissues that express the NIS symporter.

It is desirable, therefore, to have new methods and compositions for more efficiently transporting iodide and other NIS substrates into cells and for retaining such transported substrates within the cells. In addition, it would be advantageous to have methods whereby any cell type could be made to transport and accumulate iodide intracellularly.

### SUMMARY OF THE INVENTION

The present invention provides, as a new composition of matter, modified sodium iodide symporter (NIS) proteins with an amino acid sequence different from that of wild-type NIS proteins. The modified NIS proteins, when expressed in a cell, result in higher intracellular concentrations of NIS substrates than does expression of the same amount of a wild-type NIS protein. The modified NIS proteins have a net electrostatic charge that is more positive than the net electrostatic charge of wild-type NIS proteins. One modified NIS protein comprises a wild-type NIS protein where one or more uncharged or negatively charged amino acids within the wild-type NIS protein are replaced by positively charged amino acids. Another modified NIS protein comprises a wild-type NIS protein that has an addition of a sequence of less than 20 positively charged amino acids at the amino terminal end, the carboxyl terminal end, or both the amino terminal and carboxyl terminal ends. Another modified NIS protein comprises a wild-type NIS protein in which a sequence of less than 20 positively charged amino acids is added internal to the amino terminal and carboxyl terminal ends of a wild-type NIS protein. Other modified NIS proteins have combinations of the above modifications (amino acid replacements and additions). The present invention also provides polynucleotides that encode the modified NIS proteins.

The present invention also provides methods for increasing the intracellular concentration of NIS substrates in a cell, methods for imaging of cells or tissues in an individual, methods for treating cancer in an individual and methods for detecting cells that have taken up a polynucleotide sequence encoding a therapeutic protein. These methods generally comprise the steps of introducing into cells, a modified NIS protein or polynucleotide encoding a modified NIS protein, then contacting the cells containing the modified NIS protein with an NIS substrate such that the NIS substrate is transported into the cells. Where the methods are used for imaging cells in an individual, the NIS substrates taken up by the cells containing modified NIS protein are capable of being imaged by scintigraphic or other methods. Where the methods are used for treating cancer in an individual, the NIS substrates taken up by the cells containing modified NIS proteins are capable of reducing viability or decreasing growth rate of the cells.

### BRIEF DESCRIPTION OF THE DRAWINGS

The present invention may be more readily understood by reference to the following drawings wherein:

FIG. 1 is the polynucleotide sequence of the coding strand of the wild-type human NIS coding sequence (accession number U66088) (SEQ ID NO. 1).

FIG. 2 is the amino acid sequence of the wild-type human NIS protein (SEQ ID NO. 2).

FIG. 3 is a model of wild-type human NIS protein embedded within a cellular membrane.

FIG. 4 is a retroviral construct carrying a polynucleotide encoding wild-type hNIS (human sodium iodide symporter) protein and causing increased radioactive iodine uptake in infected cells. (A) Schematic representation of the pL-hNIS-SN retroviral vector that carries the cDNA for the human sodium iodide symporter (hNIS). The hNIS cDNA was cloned downstream of the Moloney Murine Leukemia Virus long terminal repeat (LTR) promoter in the pLXSN retroviral vector. (B) In vitro radioactive iodide uptake (RAIU) of parental F98 cells (F98) and hNIS-transduced F98 cells (F98/hNIS) with or without NaClO₄. The data are presented as cpm per 1×10⁵ cells. The F98/hNIS cells showed more than a 40-fold increase in RAIU, which can be suppressed by NaClO₄, a NIS-specific inhibitor.

FIG. 5 shows hNIS expression in intracerebral F98/hNIS tumors. (A) Western blot analysis of normal rat brains, parental F98 tumors and F98/hNIS tumors showing hNIS expression only in F98/hNIS tumors. (B) Histology and hNIS immunohistochemical staining of F98/hNIS tumors. Hematoxylin and Eosin (H & E) staining of F98/hNIS brain tumors (top left). Anti-hNIS immunohistochemical staining showing F98/hNIS tumor cells infiltrating into normal brain (top right, arrows). Higher magnification of immunohistochemical staining in F98/hNIS tumors (bottom left) and parental F98 tumors (bottom right).

FIG. 6 shows ^{99m}TcO₄ scintigraphy of intracerebral F98 gliomas. (A) The images were acquired with a gamma camera using a pinhole collimator in vertex and right lateral views 20 min after tail vein injection of 2 mCi ^{99m}TcO₄. Note the visible uptake in F98/hNIS tumors in addition to the uptake in the thyroid gland and occasionally in the parotid salivary gland. The bar indicates radionuclide uptake that increases in intensity from left to right. (13) Temporal profile of ^{99m}TcO₄ scintigraphy in rats with F98/hNIS tumors imaged on different post-implantation days. Eleven days after tumor implantation, F98/hNIS tumors were detectable (arrow) at which time the tumor was measured 4.5 mm×3.8 mm. Nasal mucosa was occasionally detected as shown in the third image. This figure is representative of images for 2-3 rats/time point.

FIG. 7 shows radioiodide retention in F98/hNIS gliomas. (A) 123, scintigraphy shows radioiodide retention in F98/hNIS gliomas and thyroid glands. Thirteen days post-implantation with F98/hNIS cells, rats were injected with 250 μCi ¹²³I via the tail vein, and images were acquired at different time points after ¹²³I injection. Top panel shows the images acquired by a pinhole collimator in vertex view. Bottom panel shows the whole body images acquired simultaneously by a planar collimator in ventral view. Arrows on right of each panel indicate ¹²³I uptake in brain tumors while arrows on left indicate the uptake in thyroids. N; nasal mucosa, S; stomach, B; bladder. Note that the distance from thyroid to tumor on different images may vary depending on the position of animal subject, as the thyroid and brain tumor are not at the same plane. Bar indicates radionuclide uptake that increases in intensity from left to right. This figure is representative of images 3 rats/time point. (B) Mean retention of ¹²³I in 3 rats with F98/hNIS tumors. The graph was generated using the geometric means of regions of interest (ROIs) of the tumors, thyroids, and shoulder regions (as a background activity (Bkg) to investigate the general clearance of radioactivity) in two views (vertex and then ventral) of pinhole images for each different time point. ¹²³I uptake was seen in F98/hNIS gliomas through 24 hr ¹²³I post-injection.

FIG. 8 shows reduced NIS expression/function by thyroxine-supplemented diet. (A) ^{99m}TcO₄ scintigraphy of thyroids in rats fed either a normal diet or a T4-supplemented diet. The thyroid gland was undetectable by ^{99m}TcO₄ scan in rats fed a T4-supplemented diet for 11 days. The location of the thyroid is indicated by an arrow. Bar indicates radionuclide uptake that increases in intensity from left to right. N: nasal mucosa, S: salivary, T: thyroid. (13) Western blot analysis for endogenous rat NIS glycoprotein in the thyroid glands of rats fed a T4-supplemented diet for 11 days (Thy-T4) or a normal diet (Thy-ND). Note that NIS expression level was greatly reduced in rats fed T4-supplemented diet (Thy-T4). PNGase-F was used to deglycosylate NIS.

FIG. 9 shows increased survival time in ¹³¹I-treated rats bearing F98/hNIS glioma. Survival curves are shown for rats bearing F98/hNIS gliomas with and without ¹³¹I treatment and F98/LXSN gliomas with ¹³¹I treatment.

FIG. 10 is a schematic diagram of polynucleotides encoding different NIS proteins. The polynucleotide pictured at the top of the figure, FL-hNIS, encodes the open reading frame (ORF) for the NIS protein and also contains non-translated regions at both the 5′ and 3′ ends of ORF. The polynucleotide pictured in the middle of the figure, ORF-hNIS, encodes the ORF for NIS protein but lacks the non-translated regions. The polynucleotide pictured at the bottom of the figure, ORF-hNIS-(lys)₁₀, encodes the ORF of the NIS protein, which here also encodes a continuous sequence of 10 lysine amino acids at the 3′ end of the protein.

FIG. 11 is a diagram showing the polymerase chain reaction (PCR) strategy used to add the sequence encoding 10 lysine amino acids to the 5′ end of the gene. The F1B and R9 primers amplify the ORF of hNIS. To add lysine residues to the C-terminal end of the protein, the 5′ end of the R9 primer was modified to contain a sequence complementary to a sequence encoding ten consecutive codons for lysine. The resulting primer, R9-(lys)₁₀ (SEQ ID NO. 3), was used with the F1B primer to amplify sequences containing the hNIS ORF and to obtain ORF hNIS-(lys)₁₀, as shown. At the bottom of the figure, the DNA sequence of the R9-(lys)₁₀ primer is shown which illustrates codons encoding 10 consecutive lysine residues at the C-terminal end of the hNIS protein. The amino acids sequence of 10 lysines, KKKKKKKKK, is SEQ ID NO. 4.

FIG. 12 is a graph showing the results of an RAIU assay of cells containing control plasmid DNA (pcDNA3), plasmid DNA encoding the hNIS ORF (pcDNA3/ORF-hNIS), or plasmid DNA encoding a hNIS gene with 10 lysine amino acid residues inserted at the C-terminus of the hNIS protein.

### DETAILED DESCRIPTION OF THE INVENTION

In accordance with the present invention, modified NIS proteins and polynucleotides encoding modified NIS proteins are provided. The modified NIS proteins have a net electrostatic charge that is more positive than the net electrostatic charge of the wild-type NIS protein from which the modified NIS protein is derived. Expression of the modified NIS proteins in cells results in increased uptake and intracellular retention of NIS substrates as compared to cells expressing equivalent amounts of wild-type NIS proteins. The modified NIS proteins and polynucleotide sequences are particularly advantageous for use in methods described herein for imaging cells and treating cancer in an individual. The methods provide for introducing a modified NIS protein or polynucleotide encoding a modified NIS protein, into the cells in an individual that are to be imaged or treated. The cells in which the modified NIS protein is expressed are then contacted with one or more NIS substrates. NIS substrates suitable for imaging are used if the cells are to be imaged. NIS substrates that are cytotoxic or are able to inhibit cellular proliferation are used if therapeutic treatment for cancer is intended. The NIS substrates are transported into the cells expressing the modified NIS protein.

NIS Proteins and Polynucleotide Sequences

The Na⁺/I⁻ symporter (NIS) is a plasma membrane protein that mediates active iodide (I⁻) uptake into thyroid follicular cells. NIS is likely a glycoprotein. NIS also transports pertechnate (TcO₄⁻)²⁷, perrhenate (ReO₄⁻)^{27,28}, and astatide (At⁻) into cells. The molecules transported intracellularly by wild-type NIS protein or modified NIS protein are herein called “NIS substrates.” The cells of a variety of species contain polynucleotide sequences encoding NIS proteins. In rat, the gene encoding NIS encodes a protein of 618 amino acids with a predicted molecular mass of 65.2 kDa (rNIS). In humans, the gene encoding NIS encodes a protein of 643 amino acids with a predicted molecular mass of 68.7 kDa (hNIS). NIS proteins from human, rat, and other species can be used in the modified NIS proteins described herein. The NIS proteins normally found in human, rat and other species are referred to as “wild-type” NIS proteins, or herein just as “NIS proteins.”

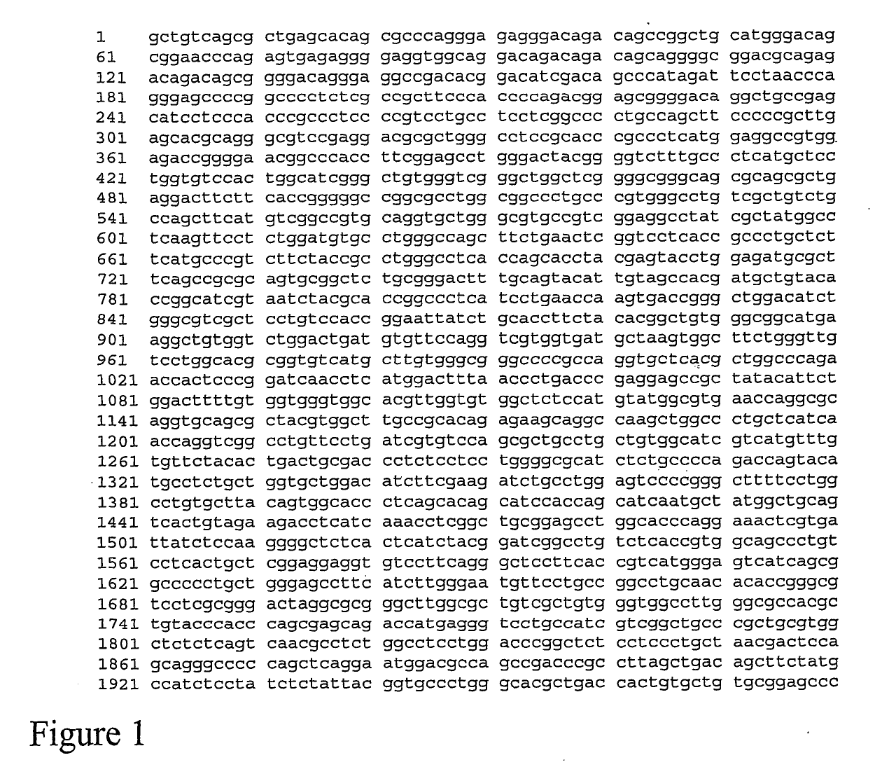

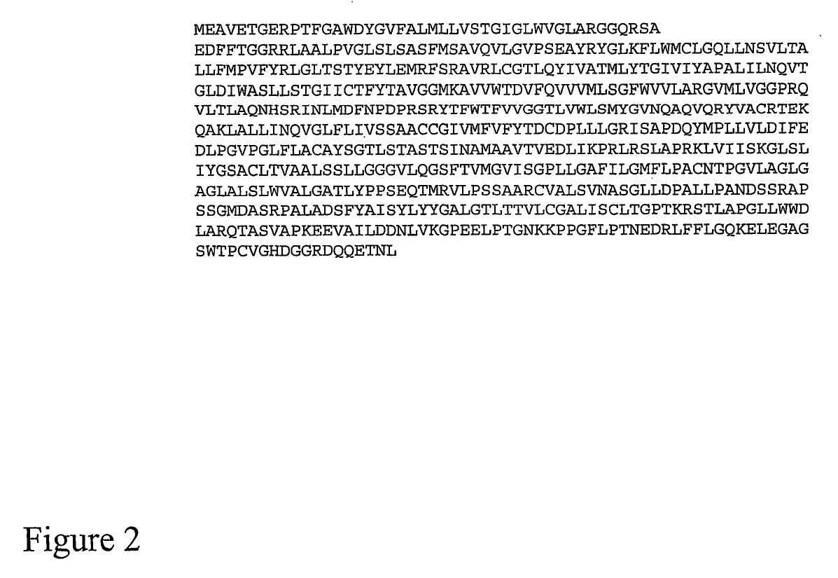

FIG. 1 shows the polynucleotide sequence encoding wild-type NIS protein from human (hNIS) (SEQ ID NO. 1). FIG. 2 shows the amino acid sequence of the human wild-type NIS protein (SEQ ID NO. 2), encoded by the polynucleotide sequence in FIG. 1. The amino acid sequence encoded by hNIS has 84% identity and 92% similarity to rat NIS protein (rNIS), with a 5 amino acid insertion in the loop between the last two hydrophobic domains and a 20 amino acid insertion in the carboxy terminus.

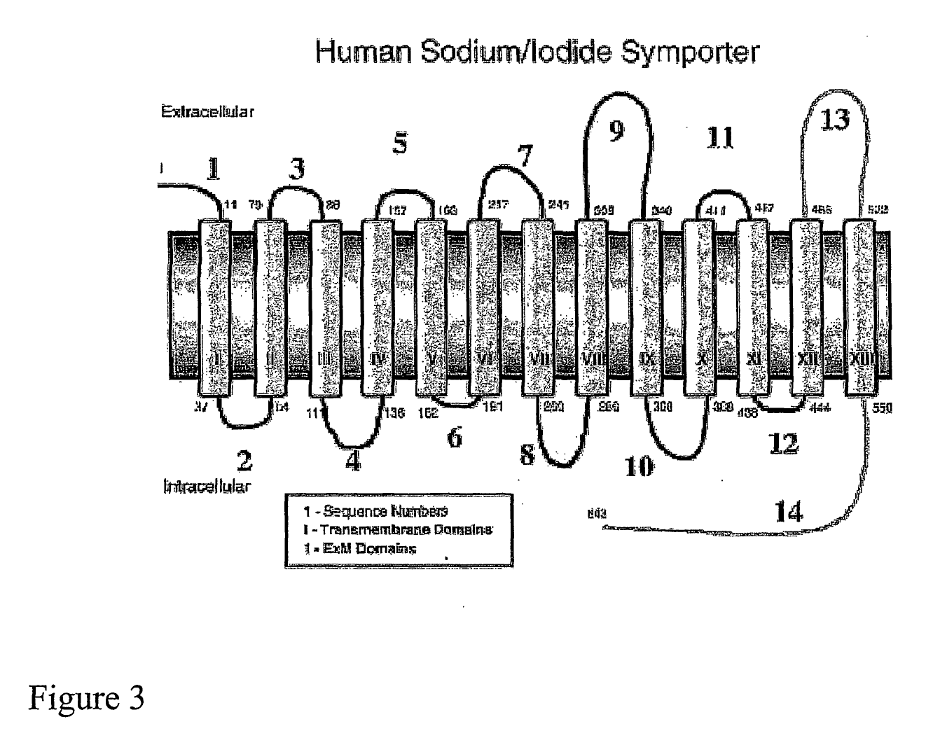

The NIS protein is a membrane protein (see FIG. 3) and has as its function, transport of NIS substrates into the cell. As part of the cellular plasma membrane, the NIS protein has 13 transmembrane helices. There are 13 regions of the protein that are embedded within the cellular membrane (i.e., transmembrane regions). There are also regions of the protein that are not embedded within the membrane (i.e., extra-membrane regions). At one end of each of 13 transmembrane regions is a part of the protein that is located outside the cell. These parts of the protein are herein referred to as extra-membrane regions or domains that are located on the extracellular side of the membrane. These regions or domains are also referred to as extracellular extra-membrane domains. At the other end of each of the 13 transmembrane regions is another part of the protein that is located inside the cell. These parts of the protein are herein referred to as extra-membrane regions or domains that are located on the intracellular side of the membrane. These regions or domains are also referred to as intracellular extra-membrane domains. Additionally, the amino terminus of the NIS protein is located on the extracellular side of the membrane and the carboxyl terminus of the protein is located on the intracellular side of the membrane.^{31,32} This structural model of the NIS protein is shown in FIG. 3.

The NIS protein is the molecular basis for using radioiodide as a scintigraphic imaging and therapeutic agent for tissues showing iodide uptake (e.g., thyroid cells). Cells expressing NIS protein transport NIS substrates into their interior (i.e., intracellularly). Such transport results in intracellular concentrations of the NIS substrates. The NIS substrates can be of a variety of types. The iodide, or other substrate, can be isotopic, meaning that it is or is radioactive or is associated with radioactivity. The iodide, or other substrate, can also be labeled by means other than radioisotopically. The NIS substrate is chosen depending on the intended purpose of the method. If the method is used therapeutically for cancer, the NIS substrate preferably has cytotoxic activity or the ability to inhibit cell proliferation. If the method is used for imaging, the NIS substrate preferably has some type of label that is detectable by scintigraphic methods. Such imaging techniques are normally used diagnostically or prognostically for detecting the presence, size, metastasis and other properties of a tumor or cancer in a patient. Still another use of cell uptake of isotopic or non-isotopic and labeled radioactive NIS substrates is for determining the ability of specific cells to transport NIS substrates intracellularly, as in the case where cells have a defect in this process, for example.

Modified NIS Proteins and Polynucleotide Sequences

Herein, “modified” NIS proteins refers to proteins that function as do wild-type NIS proteins, to transport NIS substrates from outside of cells to inside of cells, but whose expression results in higher intracellular concentrations of NIS substrates than does equivalent expression levels of wild-type NIS proteins. Herein, modified NIS proteins have an increased net positive electrostatic charge or a more positive net electrostatic charge as compared to the wild-type NIS protein from which the modified NIS protein was derived (i.e., the sum of all charge contributions from all amino acids comprising the modified NIS protein is more positive than that for the wild-type protein from which the modified protein is derived). Electrostatic charge of proteins is determined by methods known in the art. Since the charge of individual amino acids at a specific pH is known, one method for determining charge of a protein of known amino acid sequence at a specific pH is to sum all of the individual charges of the amino acids that comprise the protein. Other methods exist for determining the electrostatic charge of a protein. Isoelectric focusing is one experimental method that can be used to approximate the electrostatic charge of a protein.

The modified NIS proteins, when expressed at a specific concentration in a cell, result in higher intracellular levels of one or more NIS substrates than does expression in a similar cell of the wild-type NIS protein. While not wishing to be bound by a mechanism, it is thought that the increased intracellular levels of NIS substrates due to expression of modified NIS proteins could come about for a variety of reasons, including an increased transport rate of NIS substrates into cells, an increased intracellular retention time of NIS substrates or an increased efficiency of cellular localization of modified NIS protein.

Modified NIS proteins have an amino acid sequence that is different from the amino acid sequence of wild-type NIS protein. The increased positive charge of modified NIS proteins is due to the amino acid sequence of modified NIS proteins having greater numbers of positively charged amino acids and/or fewer numbers of negatively charged amino acids than the amino acid sequences of corresponding wild-type NIS proteins. Positively charged amino acids are amino acids that have a net positive charge at a given pH, normally at around pH 7.0. Herein, positively charged amino acids are arginine, lysine and histidine. Negatively charged amino acids are amino acids that have a net negative charge at a given pH, normally at or around pH 7.0. Herein, negatively charged amino acids are aspartic acid and glutamic acid. Other amino acids are herein referred to as uncharged amino acids. Uncharged amino acids are alanine, asparagine, cysteine, glutamine, glycine, isoleucine, leucine, methionine, phenylalanine, proline, serine, threonine, tryptophan, tyrosine and valine.

The increase in the net positive electrostatic charge of modified NIS proteins, as compared to wild-type NIS proteins, can be brought about in a variety of ways. In one method, one or more positively charged amino acids are added to the amino acid sequence of a wild-type NIS protein. “Addition” of amino acids means that the amino acids present in the wild-type NIS protein are still present (i.e., there is no substitution, replacement or deletion of these amino acids). In one embodiment, positively charged amino acids are added to the amino terminal end of the amino acid sequence of a wild-type NIS protein, to the carboxyl terminal end of the amino acid sequence of a wild-type NIS protein, or to both the amino terminal and carboxyl terminal ends of the amino acid sequence of a wild-type NIS protein.

In another embodiment, positively charged amino acids are added to the wild-type NIS protein at one or more locations internal to the amino terminal and carboxyl terminal ends of the wild-type protein. The amino acids can be added to amino acid sequences of the wild-type NIS protein that are located within the cellular membrane (i.e., the transmembrane domain). There are thought to be 13 such transmembrane regions that span the cellular plasma membrane (see FIG. 3). The internally added amino acids can also be added to amino acid sequences of the wild-type NIS protein that are located external to the cellular membrane. Such external or extra-membrane domains are also seen in FIG. 3. Extra-membrane domains are of two types. Extra-membrane domains can be located on the extracellular side of the membrane. Extra-membrane domains can also be located on the intracellular side of the membrane. Positively charged amino acids can be added to extra-membrane domains located either on the extracellular or intracellular side of the membrane. Preferably, positively charged amino acids are added to extra-membrane domains of the wild-type NIS protein. More preferably, the positively charged amino acids that are added to extra-membrane domains are added to intracellular extra-membrane domains.

Positively charged amino acids can be added to the amino acid sequence of a wild-type NIS protein as single amino acids. A single positively charged amino acid can be added to the amino terminal end, carboxyl terminal end, or both the amino terminal end and carboxyl terminal end of a wild-type NIS protein.

Although single positively charged amino acids can be added to a wild-type NIS amino acid sequence, it is preferable that a plurality of positively charged amino acids are added as a continuous sequence of amino acids that itself has a net positive charge. Such a sequence of amino acids has one or more positively charged amino acids but may also have uncharged amino acids or even negatively charged amino acids, as long as the net charge of the continuous sequence itself is positive.

The continuous sequence of amino acids is attached to the amino acid sequence of a wild-type NIS protein through one or two peptide bonds; one peptide bond if the continuous sequence of amino acids is attached to either the amino terminal or carboxyl terminal end of the wild-type NIS protein, and two peptide bonds if the continuous sequence of amino acids is attached to the wild-type NIS protein internal to the amino and carboxyl terminal ends of the wild-type protein. It is possible to have a modified NIS protein where more than one continuous sequence of amino acids is added to the amino acid sequence of a wild-type NIS protein.

Preferably, the continuous sequence of amino acids that has a net positive charge is a sequence of amino acids all of which are positively charged. In such a sequence, there are consecutive positively charged amino acids attached to one another through peptide bonds, with no intervening amino acids that are not positively charged. Herein, such a sequence is called a sequence of continuous positively charged amino acids.

Herein, the sequence of continuous positively charged amino acids can be from between 2 amino acids in length to 19 amino acids in length (i.e., less than 20 amino acids in length). The sequence of continuous positively charged amino acids can be of length 2, 3, 4, 5, 6, 7, 8, 9, 10, 11, 12, 13, 14, 15, 16, 17, 18 or 19 amino acids in length. Such sequences can contain multiples of a single positively charged amino acid. For example, given that arginine is represented by “R,” lysine is represented by “K” and histidine is represented by “H,” such a sequence can be (R)₁₉ (SEQ ID NO. 5) or (K)₁₀ (SEQ ID NO. 4), or any number of other possibilities. Such sequences can contain two different positively charged amino acids. Examples are RKKKRRRKRKR (SEQ ID NO. 6), HK, KRRRRRRRRRRRRRRRK (SEQ ID NO. 7), or any of a number of other possibilities. Such sequences also can contain all three positively charged amino acids. For example, RKH, HRKKKKKKKRHHH (SEQ IID NO. 8), RRRRRRRHK (SEQ ID NO. 9), or any of a number of other possibilities are possible.

Addition of single positively charged amino acids or of continuous sequences that have a net positive charge to the amino acid sequence of a wild-type NIS protein normally increases the total length (i.e., the total number of amino acids comprising) of the protein. However, it is possible to delete amino acids from a wild-type NIS protein and then add the positively charged amino acids to the protein. In such a case, the added positively charged amino acids can replace amino acids at the same location in the wild-type protein (see below). It is also possible that wild-type amino acids at one location in the wild-type protein are removed and the positively charged amino acids are added at a different location within the wild-type protein. In such cases, the resulting modified NIS protein may not be longer than a wild-type NIS protein.

However the positively charged amino acids are added to the amino acid sequence of a wild-type NIS protein, the resulting modified NIS protein retains the ability to transport NIS substrates into a cell. Preferably, expression of a modified NIS protein in a cell results in a higher intracellular concentration of NIS substrates than equivalent expression of a wild-type NIS protein.

In another method for obtaining a modified NIS protein by increasing the net positive charge of a wild-type NIS protein, one or more amino acids in a wild-type NIS protein that are not positively charged (i.e., are negatively charged or are uncharged) are substituted with or replaced by one or more positively charged amino acids. The amino acids that are replaced can be amino acids that are at the amino terminal end, the carboxyl terminal end or both the amino terminal and carboxyl terminal ends of a wild-type NIS protein. The amino acids that are replaced can also be amino acids that are located internal to the amino and carboxyl terminal ends of the wild-type NIS protein. These internal amino acids that are replaced can be located in the transmembrane domains or the extra-membrane domains of the wild-type NIS protein. Preferably, the amino acids that are replaced are located in the extra-membrane domain. More preferably, the amino acids that are replaced are located in extra-membrane domains that are located on the intracellular side of the membrane.

It is also possible to substitute or replace a negatively charged amino acid in a wild-type NIS protein with an uncharged amino acid in order to increase the net positive electrostatic charge of wild-type NIS protein. The negatively charged amino acid can be located in any region of the wild-type NIS protein in which non-positively charged amino acids, described above, are located. In addition, a modified NIS protein can be made by deleting negatively charged amino acids from the sequence of a wild-type NIS protein, without replacement or addition of other amino acids.

A modified NIS protein made by the substitutions or replacements described above retains function to transport NIS substrates into cells, and preferably possesses improved function, as measured by increased intracellular concentration of NIS substrates.

It should be recognized that a modified NIS protein can also be made using combinations of any of the above methods. For example, a modified NIS protein can have one or more additions of positively charged amino acids to a wild-type NIS protein and one or more replacements of uncharged or negatively charged amino acids with positively charged amino acids. A modified NIS protein can have one or more additions of positively charged amino acids and deletions of one or more negatively charged amino acids from the sequence of a wild-type NIS protein. A modified NIS protein can have one or more replacements of uncharged or negatively charged amino acids in a wild-type NIS sequence with positively charged amino acids and deletions of one or more negatively charged amino acids. A modified NIS protein can also have one or more additions of positively charged amino acids, one or more replacements of uncharged or negatively charged amino acids in a wild-type NIS sequence with positively charged amino acids, and deletions of one or more negatively charged amino acids from the sequence of a wild-type NIS protein.

A polynucleotide sequence that encodes wild-type NIS is altered to encode a modified NIS protein using various biochemical and molecular biological methods, such as recombinant DNA methods, to produce a polynucleotide sequence that encodes the desired modified NIS protein. For example, a polynucleotide encoding a modified NIS protein can be made using the polymerase chain reaction (PCR) as a site-directed mutagenesis method. This technique allows for deleting amino acids from a polynucleotide sequence encoding a wild-type NIS protein, adding amino acids to the wild-type sequence, or substituting amino acids in the wild-type sequence. In one example of PCR based site-directed mutagenesis, a DNA molecule encoding a wild-type NIS amino acid sequence is used as a template for the PCR. Vector-specific primers and oligonucleotide primers designed to encode the changes, i.e., the deletions, additions, or substitutions sought to be introduced into the wild-type sequence, are used during amplification to provide DNA molecules containing the desired polynucleotide changes. Polynucleotide molecules containing the polynucleotide encoding the modified NIS protein are isolated from the mixture of PCR products. Use of PCR to produce a polynucleotide encoding one specific modified NIS protein is described in Example 7.

As recognized by one of skill in the art, a specific modified NIS protein can be encoded by a variety of polynucleotides due to the fact that, generally, more than one codon encodes a single amino acid. To encode a specified amino acid, therefore, it may be possible for any one, or any combination of the codons encoding that specific amino acid to be used in a polynucleotide that encodes a specific modified NIS protein.

Normally, modified NIS proteins are made through expression (i.e., transcription and translation) of a polynucleotide sequence encoding the modified NIS protein. Expression of a cloned gene, such as a polynucleotide containing a gene encoding a modified NIS protein, can be achieved in a variety of cell types and then the expressed protein can be purified from the cells. Such techniques are well known in the art. Among cell types that are used for expression of cloned genes are various species of bacterial cells, yeast cells and insect cells. The genes encoding the proteins are normally introduced into the cells using one of a variety of types of expression vectors. After introduction of the gene into the cells and expression therein of the protein encoded by the gene, various biochemical methods are used to purify the desired protein from the cells. Such techniques are also well known in the art.

Vectors Expressing Modified NIS Protein

Normally, modified NIS proteins are made through expression (i.e., transcription and translation) of a polynucleotide sequence encoding the modified NIS protein. In the methods of the present invention, a polynucleotide sequence encoding a modified NIS protein is expressed in cells. As described above, the modified NIS protein may be expressed in a type of cell (e.g., bacterial, yeast, insect) which facilitates purification of the expressed protein from the cell. In another type of expression, the modified NIS protein is expressed in cells of an individual for the purposes of facilitating therapy or imaging. In these methods, the polynucleotides encoding the modified NIS proteins are expressed in the cells and the modified NIS proteins are produced. The modified NIS proteins are localized to, and become part of, the plasma membrane of the cells in the individual (see FIG. 3).

In order to introduce the polynucleotide sequence encoding the modified NIS protein into cells, the protein coding region of the polynucleotide is normally attached to sequences that facilitate its transcription into mRNA as well as translation of the mRNA into modified NIS protein. A strategy common in the art for doing this is to clone the polynucleotide sequence encoding the modified NIS protein into an expression vector which contains sequences facilitating transcription and translation of the cloned polynucleotide sequence.

In the art, “vectors” refers to nucleic acid molecules capable of mediating introduction of another nucleic acid or polynucleotide sequence to which it has been linked into a cell. One type of vector is an episome, i.e., a nucleic acid capable of extrachromosomal replication. Other types of vectors become part of the genome of the cell into which they are introduced. Vectors capable of directing the expression of inserted DNA sequences are referred to as “expression vectors” and may include plasmids, viruses, or other types of molecules known in the art. Other types of vectors are plasmid vectors.

Expression vectors normally contain sequences that facilitate gene expression. An expression vector can comprise a transcriptional unit comprising an assembly of a protein encoding sequence and elements that regulate transcription and translation. Transcriptional regulatory elements generally include those elements that initiate transcription. Types of such elements include promoters and enhancers. Promoters may be constitutive, inducible or tissue specific. Transcriptional regulatory elements also include those that terminate transcription or provide the signal for processing of the 3′ end of an RNA (signals for polyadenylation). Translational regulatory sequences are normally part of the protein encoding sequences and include translational start codons and translational termination codons. There may be additional sequences that are part of the protein encoding region, such as those sequences that direct a protein to the cellular membrane, a signal sequence for example.

The expression vectors described herein may contain tissue-specific promoters driving transcription of the modified NIS polynucleotide sequence. The present expression vectors also may contain inducible promoters that drive transcription of the modified NIS polynucleotide sequence. There are a variety of tissue-specific and inducible transcriptional regulatory sequences known in the art. Any of these sequences can be used. The use of such promoters is advantageous in that it is desirable for both gene therapy and diagnostic imaging based on introduction of modified NIS polynucleotide sequences, to have the polynucleotides encoding the modified NIS genes express the encoded proteins in specific cells. One way to obtain such cell-type specific expression is to introduce the polynucleotide encoding the modified NIS protein only into such cells. Another way to obtain cell-type specific expression is to introduce the polynucleotide encoding the modified NIS gene into all or many cells, then have a way to express (i.e., transcribe) that introduced gene in only the desired target cells. In the cells that are not the desired targets, the gene is not expressed (i.e., transcribed). This latter method often can be accomplished using tissue-specific transcriptional promoters in the vector, that drives transcription only in cells in which the particular tissue-specific promoter is active.

Typically, vectors contain one or more restriction endonuclease recognition sites which permit insertion of the polynucleotide encoding the modified NIS protein The vector may further comprise a marker gene, such as for example, a dominant antibiotic resistance gene, which encodes proteins that serve to identify and separate transformed cells from non-transformed cells.

One type of vector used is a viral vector. Viral vectors are recombinant viruses which are generally based on various viral families comprising, for example, poxviruses, herpesviruses, adenoviruses, parvoviruses, retroviruses and others. Such recombinant viruses generally comprise an exogenous polynucleotide sequence (herein, modified NIS protein) under control of a promoter which is able to cause expression of the exogenous polynucleotide sequence in vector-infected host cells.

One type of viral vector is a defective adenovirus which has the polynucleotide sequence encoding modified NIS protein inserted into its genome. The term “defective adenovirus” refers to an adenovirus incapable of autonomously replicating in the target cell. Generally, the genome of the defective adenovirus lacks the sequences necessary for the replication of the virus in the infected cell. Such sequences are partially or, preferably, completely removed from the genome. To be able to infect target cells, the defective virus contains sufficient sequences from the original genome to permit encapsulation of the viral particles during in vitro preparation of the construct. Other sequences that the virus contains are any such sequences that are said to be genetically required “in cis.”

Another type of viral vector is a defective retrovirus which has the exogenous polynucleotide sequence inserted into its genome. Such recombinant retroviruses are well known in the art. Recombinant retroviruses for use in the present invention are preferably free of contaminating helper virus. Helper viruses are viruses that are not replication defective and sometimes arise during the packaging of the recombinant retrovirus.

Non-defective or replication competent viral vectors can also be used. Such vectors retain sequences necessary for replication of the virus.

Introduction of Polynucleotides Encoding Modified NIS Protein into Cells and Tissues

In one aspect, the present methods comprise introduction of polynucleotide sequences encoding modified NIS proteins, preferably contained within a vector, into specific cells so that the cells have increased levels of modified NIS protein. Such cells transport increased amounts of NIS substrates, for example, from outside cells to the interior of cells. A variety of methods can be used to introduce or transfer the polynucleotide encoding modified NIS protein into cells. One such method is known as transfection. Transfection is commonly performed using various treatments of the cells or DNA polynucleotide which facilitate uptake of the DNA by the cell. For example, cells can be treated chemically to make them permeable to DNA. DNA can also be treated, for example by containing the DNA polynucleotide within liposomes that cells can internalize. Preferably, transfection is used to introduce plasmid DNA into cells.

As described above, polynucleotides encoding modified NIS proteins can also be introduced into cells using viruses. For example, polynucleotide sequences that are to be introduced into cells are cloned into viral genomes. Infection of cells with such viruses results in introduction of the viral genome into the cell. Since the cloned polynucleotide sequence is part of the viral genome, it is introduced into the cell along with the viral genome. Such viral vectors can have DNA or RNA genomes. Numerous such viral vectors are well known to those skilled in the art. Viral vectors that have cloned polynucleotide sequences, encoding modified NIS proteins for example, cloned into their genomes are referred to as “recombinant” viruses. Transfer of DNA molecules using viruses is particularly useful for transferring polynucleotide sequences into particular cells or tissues of an animal. Such techniques are commonly known in the art as gene therapy.

Another method for introducing polynucleotide sequences into cells that are in a human or animal body involves administration of purified DNA polynucleotides encoding modified NIS protein directly into the human or animal. The polynucleotide encoding modified NIS protein preferably has attached sequences that facilitate transcription and translation once the polynucleotide is localized within cells. The administration of the DNA polynucleotides can be performed by injection of the DNA or even transfection of DNA. Such methodologies are commonly used in the vaccine field, specifically for administration of so-called “DNA vaccines.”

Expression of Modified NIS Proteins in Cells

Whatever methodology is used to administer polynucleotides encoding modified NIS proteins to human or animal individuals, such methodologies may comprise variations that result in the polynucleotide sequences being preferentially introduced into specific cells or tissues of an individual. For example, if transfer of polynucleotide sequences encoding modified NIS proteins is used for therapy of cancer or tumor cells, it is preferable that the polynucleotide sequences are introduced and/or expressed specifically in the cancer or tumor cells and less preferentially in nontumor cells. If transfer of NIS encoding polynucleotide sequences are to be used for imaging, it is preferable that the polynucleotide sequences are introduced and/or expressed specifically in cells that are to be imaged and less preferentially into cells that are not to be imaged.

There are methods known in the art of gene transfer and gene therapy for introducing exogenous polynucleotide sequences into specific cells and not into other cells. For example, techniques are known in the art that result in recombinant viruses specifically infecting certain cell types (e.g., tumor cell types) within a human or animal. For viruses, such “targeting” can be accomplished through manipulation of cellular receptors for the recombinant viruses and/or manipulation of viral ligands that recognize and bind to cellular receptors for the viruses. Such methodologies, as used to introduce a polynucleotide encoding a modified NIS protein into tumor cells in animals or humans, are within the purview of the present application. Tumor cells particularly attractive for targeting are glioblastoma multiformin and anaplastic astrocytoma. Targeting can also be accomplished by direct injection of, for example, the virus into the specific tumor.

The polynucleotide sequences encoding modified NIS proteins that are introduced into cells are preferably expressed at a high level (i.e., the introduced polynucleotide sequence produces a high quantity of modified NIS protein within the cells) after introduction into the cells. Techniques for causing a high level of expression of polynucleotide sequences introduced into cells are well known in the art. Such techniques frequently involve, but are not limited to, increasing the transcription of the polynucleotide sequence, once it has been introduced into cells. Such techniques frequently involve the use of transcriptional promoters that cause transcription of the introduced polynucleotide sequences to be initiated at a high rate. A variety of such promoters exist and are well known in the art. Frequently, such promoters are derived from viruses. Such promoters can result in efficient transcription of polynucleotide sequences in a variety of cell types. Such promoters can be constitutive (e.g., CMV enhancer/promoter from human cytomegalovirus) or inducible (e.g., MMTV enhancer/promoter from mouse mammary tumor virus). A variety of constitutive and inducible promoters and enhancers are known in the art. Other promoters that result in transcription of polynucleotide sequences in specific cell types, so-called “tissue-specific promoters,” can also be used. A variety of promoters that are expressed in specific tissues exist and are known in the art. For example, promoters whose expression is specific to neural, liver, epithelial and other cells exist and are well known in the art. Methods for making such DNA molecules (i.e., recombinant DNA methods) are well known to those skilled in the art.

After polynucleotides encoding modified NIS proteins are introduced into cells, techniques are used to determine specifically the cells into which the polynucleotide sequences have been introduced and/or the specific cells that are expressing the introduced polynucleotide sequences. A variety of techniques to examine the presence of polynucleotide sequences and/or expression of polynucleotide sequences exist and are well known in the art. Such techniques include Southern blotting, Northern blotting, polymerase chain reaction (PCR), Western blotting, RNase protection, radioiodide uptake assays, and others.

Introduction of Modified NIS Protein into Cells and Tissues

Another method involves introducing modified NIS proteins into cells. One method for introducing proteins into cells uses lipid carriers. For example, proteins that are associated with liposomes are able to enter cells when the liposomes enter or fuse with cells. Other methods of introducing proteins into cells are known. Microinjection and electroporation are two such methods.

Therapy and Imaging

Once modified NIS proteins are expressed in cells of an individual, NIS substrates are administered to the patient so that the substrates are transported into the cells. Depending on the goal of the method, different NIS substrates are used. Herein, “treatment” or “therapy” for cancer cells means that the cells are killed (i.e., the NIS substrate is cytotoxic) or the proliferation rate of the cells is decreased. Herein, “imaging” means making of a picture, image or shadow of cells or tissues such that the cells and tissues are visible and can be detected. NIS substrates used for therapy may be different than NIS substrates used for imaging.

There are a variety of known NIS substrates that can be used. In addition to iodide (r), pertechnate (TcO₄⁻), perrhenate (ReO₄⁻), and astatide (At⁻) are known NIS substrates. These substrates are generally administered to patients in chemical forms that are well known in the art. For example, I⁻ may be given to a patient as NaI. TcO⁻₄ may be administered to patients as Na⁺(TcO⁻₄).

Normally, the NIS substrates used in the methods for cancer treatment or for imaging are isotopic and include radionuclides, also called radioactive isotopes. Isotopic or radioactive is a term well known in the art that refers to the ability of a substance to emit nuclear radiation, of the type resulting from spontaneous disintegration of atomic nuclei. Isotopic iodide, for example, exists in a variety of forms. ¹²³I, ¹²⁴I, ¹²⁵I and ¹³¹I are forms of isotopic radioactive iodide known to exist. One or the other of these forms of iodine are chosen based on whether the method is used for therapy or for imaging. For example, ¹³¹I is commonly used for therapeutic purposes. ^{99m}TcO₄ is a form of isotopic pertechnate. ¹⁸⁸ReO₄ is a known isotopic form of perrhenate. ²¹¹At is a form of isotopic astatide.

Nonisotopic NIS substrates can also be used. Herein, nonisotopic NIS substrates are labeled by some means other than by incorporation of radioactivity. Nonisotopic NIS substrates are preferably used for imaging. However, isotopic NIS substrates can also be used for imaging.

A number of techniques for imaging are known in the art. Scintigraphy (e.g., Gamma camera imaging or Positron Emission Tomography (PET)) is one known group of techniques. Other imaging techniques are known in the art and can be used, such as magnetic resonance imaging (i.e., MRI), for example.

The purpose, therapy or imaging, for using the inventive method influences not only the choice of the particular NIS substrate and its form, but also influences the dosing regime of the particular substance. For example, a particular isotopic NIS substrate may be administered at a relatively higher dose if the method is used for therapy (i.e., cell killing). The same isotopic NIS substrate may be administered at a relatively lower dose if the method is used for imaging.

These NIS substrates, suitable for administration to an individual, may be administered to patients using a variety of methods well known in the art. For example, the NIS substrates may be administered intraveneously, through inhalation of an aerosol, or by direct injection into a desired tissue as in injection into a solid tumor in a patient, for example. Other methods of administering these substances are known in the art and may be used. The substances are administered using biologically effective dosages and timing of administration that are generally known in the art. Preferably, the NIS substrates are not taken up by cells that do not express an NIS protein or a modified NIS protein.

After the therapeutic or imaging methods are performed in an individual, various techniques are used to measure the effectiveness and outcome of the methods. For example, after using the method therapeutically for treatment of a particular cancer, followup studies are performed on the individual to determine if the cancer cells have been killed, for example. Likewise, use of the method for imaging cells produces an image that is viewed and whose quality may be compared to images obtained by other means or at other times.

These inventive methods preferably can be used with all cells, tissues or cancer types. Of particular interest is use of the methods for therapy or imaging of glioblastoma multiformin and anaplastic astrocytoma. The methods can be used in conjunction with other methods. For example, when the method is used for cancer treatment, additional cancer treatment methods can also be used. Likewise, the inventive method used diagnostically can be used in conjunction with other diagnostic and/or imaging methods.

Imaging Methods for Determining Expression of Other Proteins

Another method uses detection of modified NIS protein expression in cells to identify cells into which other proteins, or polynucleotides encoding other proteins, are present. In one embodiment of this method, a polynucleotide sequence encoding a therapeutic protein, for example, is cloned into an expression vector containing a polynucleotide sequence encoding a modified NIS protein. The expression vector encoding both the therapeutic protein and the modified MS protein is, for example, administered to an individual and is taken up by certain cells of the individual. In those cells that take up the expression vector, both the therapeutic protein and the modified NIS protein are expressed. An NIS substrate, suitable for use in imaging, is then administered to the individual such that it contacts the cells that are expressing the modified NIS protein, and the therapeutic protein. Expression of the modified NIS protein in the cells results in uptake of the NIS substrate into the cells. Imaging of the cells of the individual is performed to identify those specific cells that contain intracellular NIS substrate. Identification of cells containing intracellular NIS substrate is useful to determine which cells in the individual also are expressing the therapeutic protein. Such techniques may be useful, for example, in identifying cells in an individual into which a viral vector carrying a therapeutic gene has been introduced.

### EXAMPLES

The invention may be better understood by reference to the following examples, which serve to illustrate but not to limit the present invention.

### Example 1

### Recombinant Retrovirus Containing NIS used to Infect F98 Glioma Cells

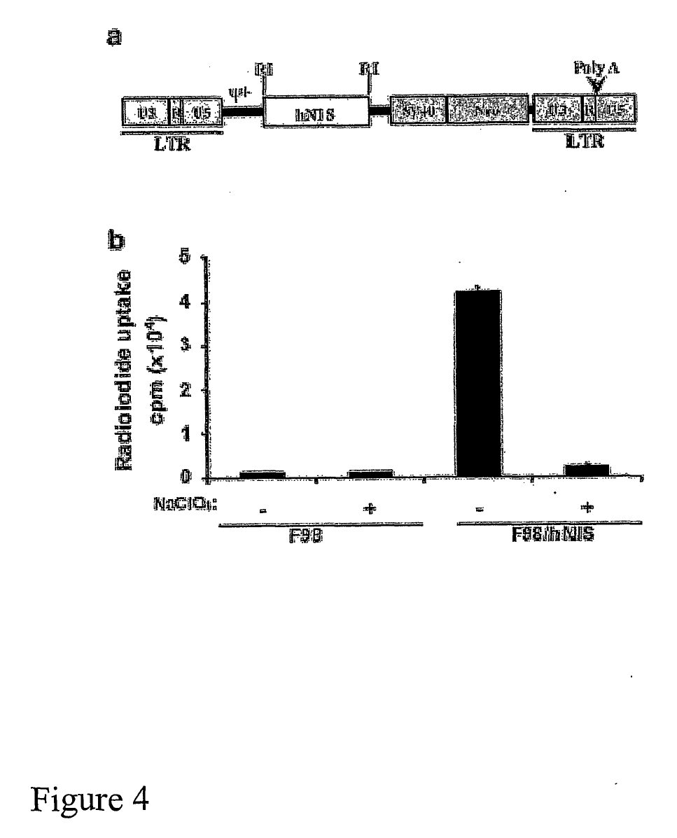

A full length wild-type hNIS polynucleotide sequence (i.e., a cDNA) was inserted into the pLXSN retroviral vector²⁰, using standard recombinant DNA techniques known to those in the art, such that the Moloney Murine Leukemia Virus Long Terminal Repeat (LTR) promoter caused hNIS expression and the SV40 promoter caused Neo^{r} expression (a construct referred to as L-hNIS-SN, see FIG. 4A).

PA317 cells (American Type Culture Collection, ATCC, Manassas, Va.) were maintained in Dulbecco's Modified Eagle's Medium (DMEM) (Gibco-BRL, Gaithersberg, MD) with high glucose and L-glutamine supplemented with 10% fetal bovine serum, 10 units/ml penicillin, and 10 μg/ml streptomycin. PA317 retroviral packing cells were transfected with 10 μg of L-hNIS-SN or LXSN DNA, respectively, using the calcium-phosphate precipitation method, as known in the art. Selection was performed using 750 μg/ml G418 (Gibco BRL) in the media for 5 days, followed by maintenance in media containing 350 μg/ml G418. Starting 11 days post-transfection, individual drug-resistant PA317/L-hNIS-SN or PA317/LXSN clones were isolated, expanded, and tested for NIS function by in vitro RAIU assay.⁵ Viral supernatant was harvested every 12 hours until cells reached 80-100% confluence, and centrifuged at 3000 g for 5 minutes at 4° C. to remove cellular debris, and stored at −80° C.

The F98 rat glioma cell line was derived from an undifferentiated neoplasm transplacentally induced by N-ethyl-N-nitrosourea in an inbred CD Fischer rat and has been propagated in vitro and in vivo since 1971. Its morphology and growth characteristics have been described in detail.¹⁷⁻¹⁹ The F98 cells were maintained in Dulbecco's Modified Eagle's Medium (DMEM) (Gibco-BRL, Gaithersberg, MD) supplemented with 10% fetal bovine serum, 2 mmol/L L-glutamine, 0.1 mM non-essential amino acids, 10 units/ml penicillin, and 10 μg/ml streptomycin (Gibco-BRL).

Ex vivo hNIS gene transfer mediated by recombinant retrovirus was performed. F98 cells were infected with the L-hNIS-SN recombinant retrovirus, using standard methods well known in the art. After G418 selection (700-750 μg/ml), individual infected clones were isolated, expanded, and maintained in media containing 350 μg/ml of G418, and then frozen in liquid nitrogen, using standard methods. Each F98/hNIS clone was tested for NIS function by an in vitro RAIU assay. One of the clones that showed >40-fold increase of RAIU activity was used for the in vivo experiments described as follows. As a negative control, F98 cells transduced with retrovirus containing the LXSN vector alone were also selected.

NIS function in the hNIS-transduced F98 glioma cells was tested in vitro by radioiodide uptake assay⁵ (RAIU). Cells (2×10⁵ cells per well) were seeded in 24-well plates. At various times after seeding, the cells were incubated for 30 min at 37° C., 5.0% CO₂ with growth media containing 2.0 μCi Na ¹²⁵I and 5-10 μM NaI carrier. The medium was aspirated and cells were quickly washed twice with ice-cold Hank's balanced salt solution (HBSS). Cells were then lysed by incubation with 95% ethanol. The radioactivity of the cell lysate was measured by a γ-counter (Packard Instruments, Downers Grove, Ill., USA). Experiments were performed in triplicate. The RAIU activity of the infected cells was expressed in terms of fold increase compared with that of mock-infected parental cells.

RAIU in hNIS-transduced F98 rat glioma cells was 40-fold higher than parental F98 cells (FIG. 4B), and the induced RAIU was inhibited by sodium perchlorate (NaClO₄), a NIS-specific inhibitor. As a negative control, F98/LXSN, F98 cells transduced with a recombinant retrovirus containing LXSN empty retroviral vector (i.e., no NIS gene), were also selected and used for subsequent animal studies of retroviral mediated gene transfer. Empty vector-transduced rat glioma cells did not induce increased RAIU.

The data show that a polynucleotide sequence encoding an NIS protein, when expressed in cells, causes cells unable to take up iodide, to transport iodide intracellularly.

### Example 2

### Intracerebral F98 Brain Tumor Model

Fischer rats weighing 175 to 200 grams (Harlan Sprague Dawley, Indianapolis, Ind.) were stereotactically implanted with tumor cells.^{11,21} The tumor cells were either F98 cells or F98 cells which had been infected with and expressed a wild-type NIS protein (see Example 1). All experimental procedures received approval from Institutional Laboratory Animal Care and Use Committee of the Ohio State University. Briefly, rats were sedated by intraperitoneal (i.p.) administration of 120 mg of ketamine/20 mg of xylazine (Fort Dodge, Fort Dodge, IA) per kg of body weight, after which a plastic screw (Arrow Machine Manufacturing, Inc., Richmond, VA) was embedded into the cranium. F98 cells were injected within 10 to 15 seconds through a central hole in the plastic screw into the right hemisphere at a concentration of 10³ (for therapeutic study) or 10⁵ (for imaging study) cells in 10 μl of serum-free DMEM containing 1.4% agarose with a gelling temperature of <30° C. The screw hole was filled with bone wax immediately following withdrawal of the needle, and the operative field was flushed with betadine before the scalp incision was closed with a single sterilized clip.

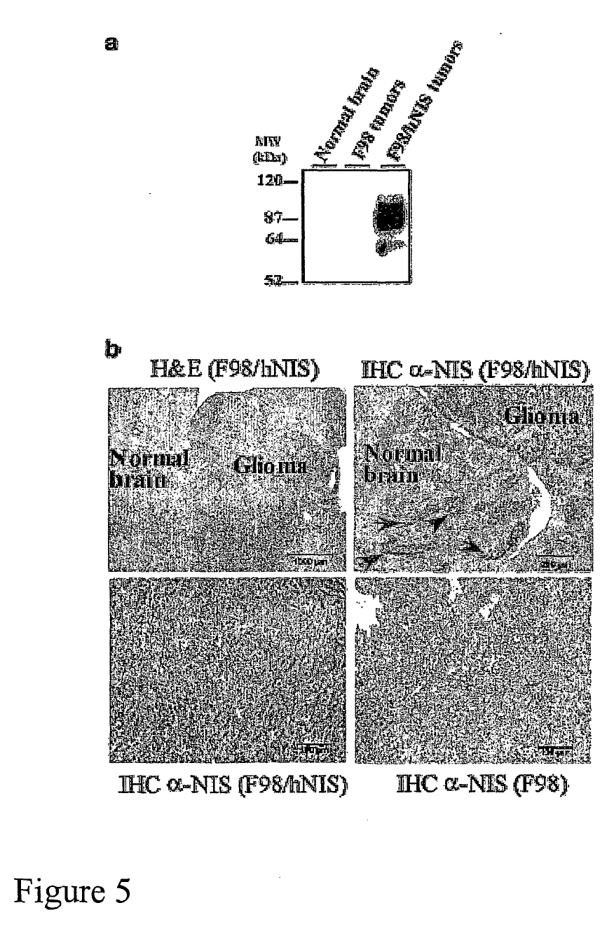

hNIS glycoprotein expression was demonstrated by Western blot analysis and immunohistochemical staining in hNIS-transduced F98 tumors after implantation and removal from the animals. Western blot analysis was performed as previously described.²³ Briefly, the tumors were removed from the animals and homogenized in a homogenizing buffer (10 mM Tris-HCl (pH 7.5), 5 mM NaCl, 1 mM EDTA, 0.1 mM PMSF, 50 μg/ml leupeptin) containing 0.25 M sucrose. The lysates were centrifuged at 700 g for 10 min at 4° C. The supernatants were further centrifuged at 200,000 g for 60-90 min at 4° C. to obtain membrane fractions, which were resuspended in homogenizing buffer without sucrose and kept at −80° C. until use. The membrane fractions (10 or 20/g per lane) were solubilized for 30 min at 37° C. in the same volume of reducing sample buffer (0.125 M Tris-HCl (pH 6.8), 4% SDS, 10% β-mercaptoethanol, 20% glycerol) and subjected to 4-20% gradient or 7.5% SDS-PAGE. The proteins were transferred on to a nitrocellulose filter which was then blocked with 5% nonfat dry mild in TBST buffer (10 mM Tris-HCl (pH 8.0), 150 mM NaCl, 0.1% Tween 20) at 4° C. overnight. Then, the transferred filter was incubated with an anti-hNIS antibody #331 (1:3,000 dilution) or anti-rNIS peptide polyclonal antibody pAb 716 (1:4,000 dilution) for 1.5 hr at room temperature, followed by incubation with peroxidase (HRP)-conjugated donkey anti-rabbit IgG (1:3,000 dilution) for 1 hr at room temperature. For deglycosylation of the proteins, denatured proteins were treated with 1 μl (500 U) of Peptide: N-glycosidase F (PNGase F) (New England Biolabs, Beverly, Mass.) in 50 mM sodium phosphate buffer (pH 7.5) containing 1% Nonidet P-40 at 37° C. for 1 hr. hNIS expression in hNIS-transduced gliomas was demonstrated by Western blot analysis with the majority of hNIS proteins detected in the 90 kDa glycosylated form (FIG. 5A).

Immunohistochemical staining²² was performed. Briefly, tissue sections were incubated with hNIS polyclonal antibodies #331 (1:1,000 dilution) at room temperature for 1 hr, and then incubated with biotinylated goat anti-rabbit IgG (1:200 dilution, Vector Lab. Inc., Burlingame, Calif.) for 20 min. The F98/hNIS tumors (FIG. 5B, top left) were similar in appearance to F98 parental tumors^{10,11} with a poorly demarcated margin from the surrounding white matter. Infiltrating tumor cells could be clearly demonstrated by immunostaining with antibody against hNIS in F98/hNIS tumors (FIG. 5B, top right). While anti-hNIS immunostaining showed extensive hNIS expression in F98/hNIS tumors (FIG. 5B, bottom left), no anti-hNIS immunostaining was found in F98 parental tumors (FIG. 5B, bottom right). The data show that F98 tumor cells into which the retroviral vector expressing NIS protein had been introduced expressed NIS protein in the animal.

### Example 3

### F98/hNIS Tumor Sizes >4.5 mm in Diameter Were Detectable By ^{99m}TcO₄ Scintigraphy

Rats were anesthetized by i.p. injection of ketamine/xylazine, and then 2.0 mCi of ^{99m}TcO₄ in 0.2 ml volume was administered via tail vein injection. Approximately 20 minutes after injection, rats were imaged with a dual head gamma camera (Picker Prism 2000, Marconi Medical Systems, Cleveland, Ohio) equipped with a pinhole collimator and a low energy ultra high-resolution parallel hole collimator (LEUHR) on each head of the gamma camera, respectively. Vertex and right lateral views with at least 500K total counts per image, were collected. Image acquisition times ranged from 2-3 minutes.

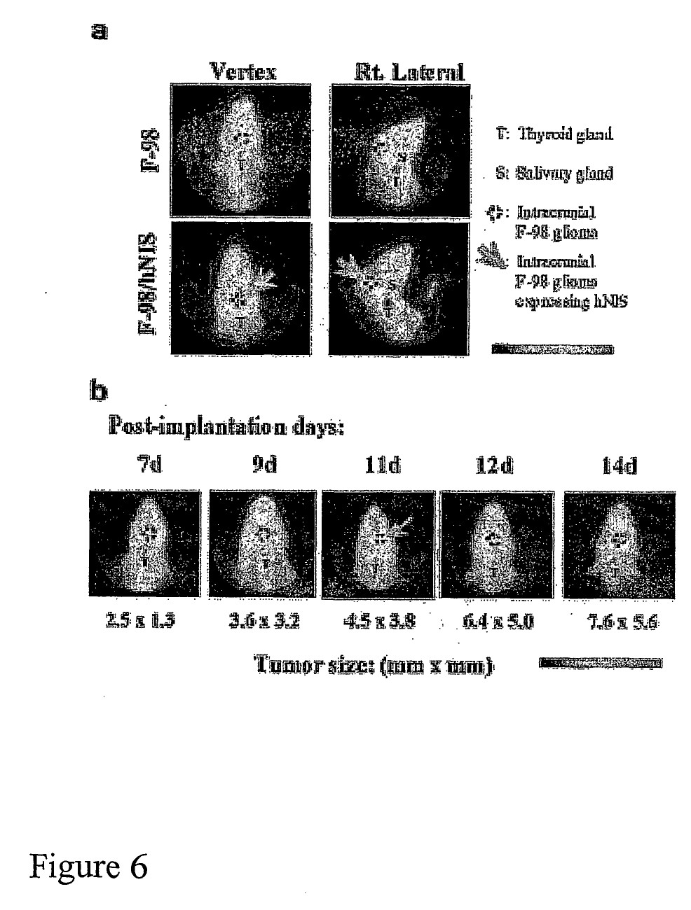

In addition to the thyroid and parotid glands, intense ^{99m}TcO₄ uptake was found in the intracerebral F98/hNIS gliomas in both vertex and lateral views (FIG. 6A). In contrast, no ^{99m}TcO₄ uptake was found in parental F98 tumors. For rats implanted with 10⁵ F98/hNIS glioma cells, the intracerebral tumor could be detected as early as eleven days after implantation (FIG. 6B), at which time the tumor measured 4.5 mm×3.8 mm in the largest tumor area at postmortem examination. The data show that cells in an animal that express NIS protein can be imaged using scintigraphic techniques.

### Example 4

### ¹²³I is Retained in the F98/hNIS Tumors up to 24 Hours

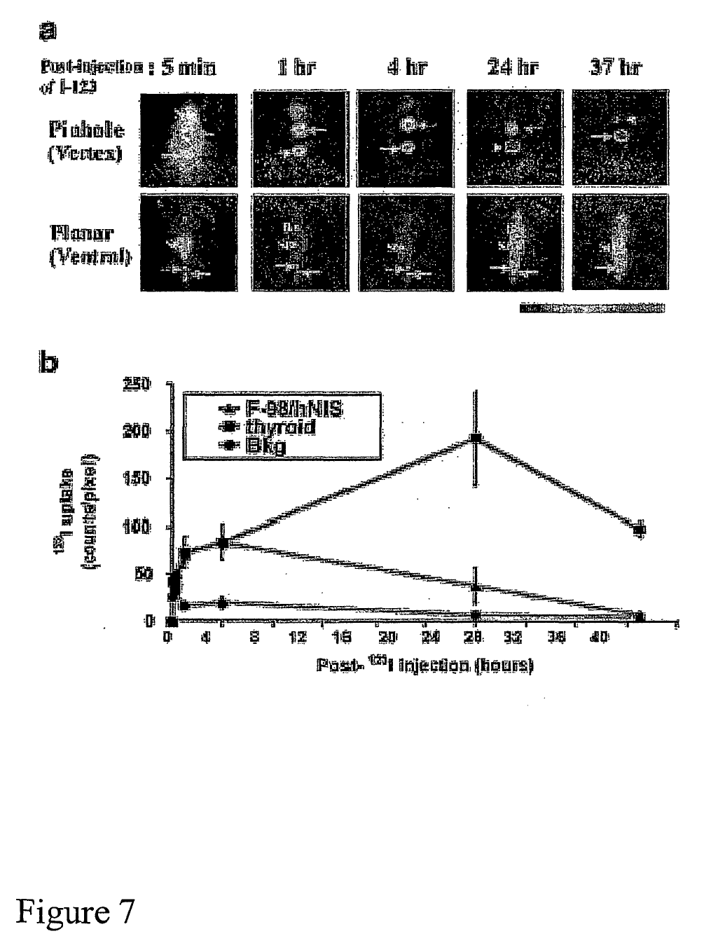

Serial ¹²³I images were obtained to determine the retention of radioiodide in the hNIS-transduced tumors (FIG. 7A). The average survival time for rats implanted with 10⁵ F98/hNIS glioma cells was about 14-16 days. For studies of ¹²³I retention, images were acquired on days 13 to 15 following tumor implantation at which time ¹²³I (0.25 mCi) was administered via tail vein injection. Modified conjugate images were obtained sequentially with the rat first in dorsal-ventral and then ventral-dorsal positions on the top of the LEUHR collimator at 5 min, 20 min, 1 hr, 4 hr, 24 hr, and 37 hr following injection. The acquisition time at 5 min post-injection image was 5 min, and the acquisition times at subsequent time points were corrected for the physical decay of ¹²³I. Regions of interest (ROI) were drawn over the tumor, thyroid, and shoulder region (as a background) in two views (vertex and then ventral) of pinhole images for each different time point, and the geometric means of the count rates (counts/pixel) for each ROI in each view were calculated. Biological half-life of radioiodine was estimated based on the time activity curve generated by plotting the tumor ROI count rates versus time.

Top panels in FIG. 7A show the serial ¹²³I images acquired by pinhole collimator in vertex view, and the bottom panels show the simultaneous images acquired with a planar collimator in ventral view. Radioiodide uptake was evident in F98/hNIS gliomas through 24 hrs post-injection. However, ¹²³I uptake was barely detectable in F98/hNIS gliomas by 37 hours post-injection. Based on the time activity curve shown in FIG. 7B, the biological half-life of ¹²³I in F98/hNIS gliomas was estimated to be 10 hours. In comparison, the biological half-life of ¹²³I in the thyroid was estimated to be greater than 20 hours.

### Example 5

### RAIU and NIS Expression in the Rat Thyroid was Reduced by Thyroxin (T4) Supplementation

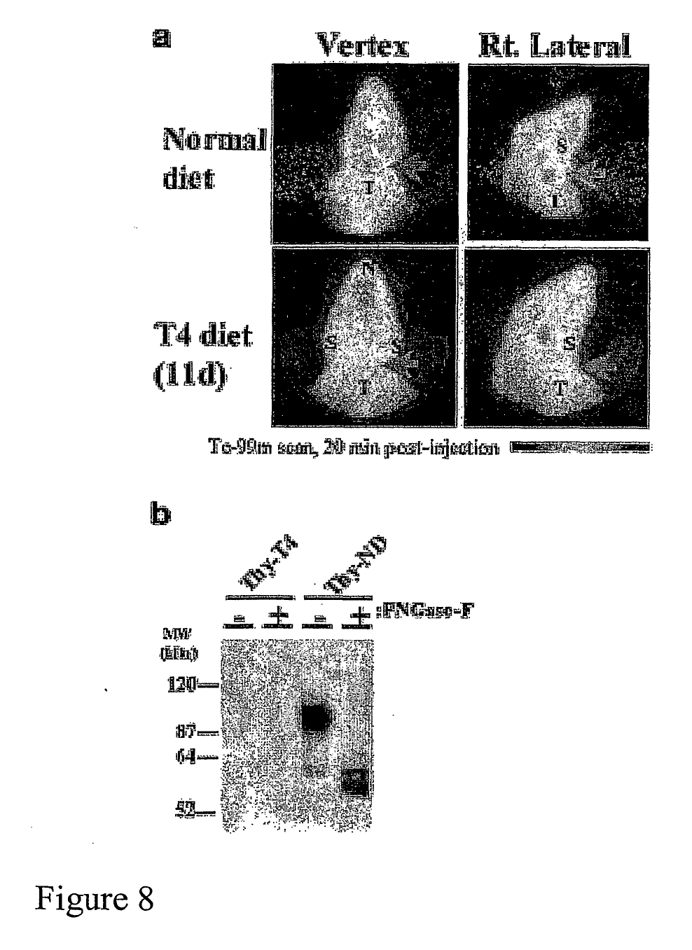

Rats fed a T4-supplemented diet were expected to have reduced RAIU in thyroid, as the endogenous NIS expression and activity in thyroid tissues is TSH-dependent. As shown in FIG. 8A, thyroid was not detectable in rats fed a T4-supplemented diet for 11 days. In comparison, thyroid was readily detectable by ^{99m}TcO₄ scintigraphy in rats fed a normal diet. By Western blot analysis, performed as described in Example 2 above, NIS glycoprotein was detected in the thyroids from rats fed a normal diet, but barely detected in the thyroids from rats fed a T4-supplemented diet (FIG. 8B). Therefore, lack of RAIU in the thyroid gland of T4-supplemented rats was mainly due to decreased NIS protein expression.

### Example 6

### Survival was Increased in Rats with F98/hNIS Tumors by ¹³¹I Treatment

For ¹³¹I therapy studies, rats were implanted stereotatically with 1×10³ F98 cells on day 0, and the animals were divided into 3 groups: empty vector transduced tumors with ¹³¹I treatment (F98/LXSN+¹³¹I), hNIS transduced tumors without ¹³¹I treatment (F98/hNIS—¹³¹I), hNIS transduced tumors with ¹³¹I treatment (F98/hNIS+¹³¹I). Each group contained 12 rats. To minimize exposure of the thyroid gland to ¹³¹I, rats were placed on 1 ppm thyroxine (T4)-supplemented diet (Harlan Teklad, Madison, Wis.) beginning one week prior to the first day of ¹³¹I treatment. Animals received three i.p. injections, 4 mCi of ¹³¹I per injection, on days 12, 14 and 16 post-implantation for a total of 12 mCi. The tumor size index on day 14 was assumed to be ˜2 mm² based on a previous study.¹⁸ Animals were weighed three times per week. The combination of sustained weight loss, ataxia, and periorbital hemorrhage indicated that death was imminent^{21,24}. Therefore, animals displaying these signs were sacrificed, and survival times were determined from the day of tumor implantation to the day of sacrifice plus 1 day.^{21,24} The observers were not blinded to sacrifice the rats. To confirm that all animals had progressively growing tumors at the time of death, the brains were removed, fixed in 10% neutral buffered formalin, and then 2-mm coronal sections were cut with a rat brain slicer (Zivic-Miller Laboratories, Inc., Allison Park, PA), and processed for routine histopathological examination. Tumor size was measured as (the longest length)×(the length perpendicular to the longest length) in tumor sections. Survival among different experimental groups was compared using unpaired t test as well as Log-rank test. Tumor sizes among different experimental groups were compared using unpaired t test. The P value of <0.05 were considered statistically significant.

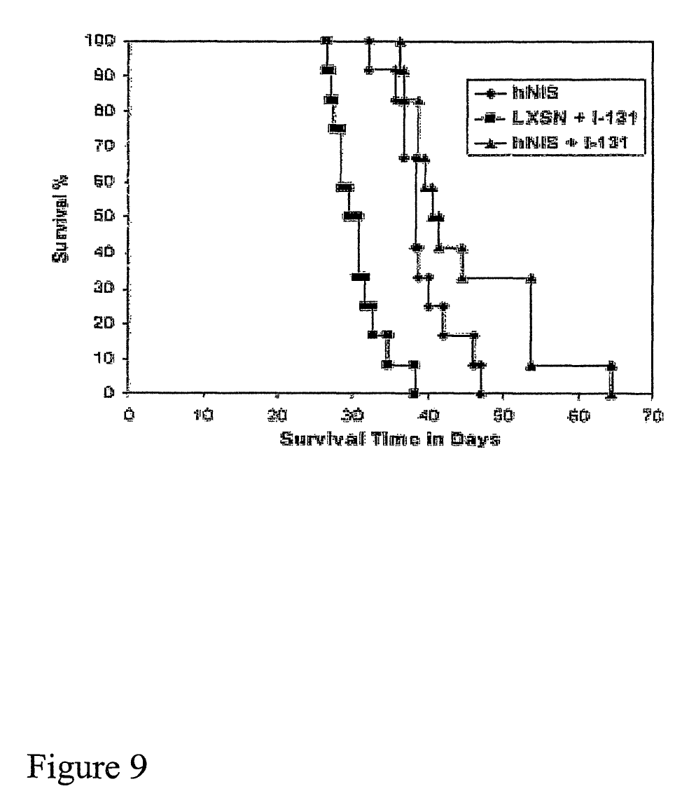

The average survival time for the animals with F98/LXSN tumors with ¹³¹I treatment, F98/NIS tumors without ¹³¹I treatment, and F98/hNIS tumors with ¹³¹I treatment were 30.4±3.2 days, 39.0±4.1 days, and 45±8.6 days, respectively (FIG. 9). The average survival time of the rats with F98/hNIS tumors with ¹³¹I treatment were prolonged compared to that of rats with F98/LXSN tumors with ¹³¹I treatment (˜2 weeks longer, P<0.01), and also compared to that of rats with F98/hNIS tumors without ¹³¹I treatment (6 days longer, P<0.05). There was no significant difference in the sizes of intracerebral glioma at the time of sacrifice among rats in the F98/LXSN with ¹³¹I treatment group (8.7±1.6 mm×6.4±1.6 mm), the F98/hNIS without ¹³¹I treatment group (8.1±2.3 mm×6.7±2.1 mm) and F98/hNIS with ¹³¹I treatment group (8.5±1.9 mm×6.1±1.5 mm). One rat in the F98/hNIS with ¹³¹I treatment group was sacrificed on day 65 due to hind limb paralysis, with a tumor size of 4.0 mm×3.5 mm. The data show that therapy with radioactive iodine resulted in increased survival of animals with tumors expressing NIS protein.

### Example 7

### Modified NIS Protein Resulted in Increased Intracellular Levels of I⁻

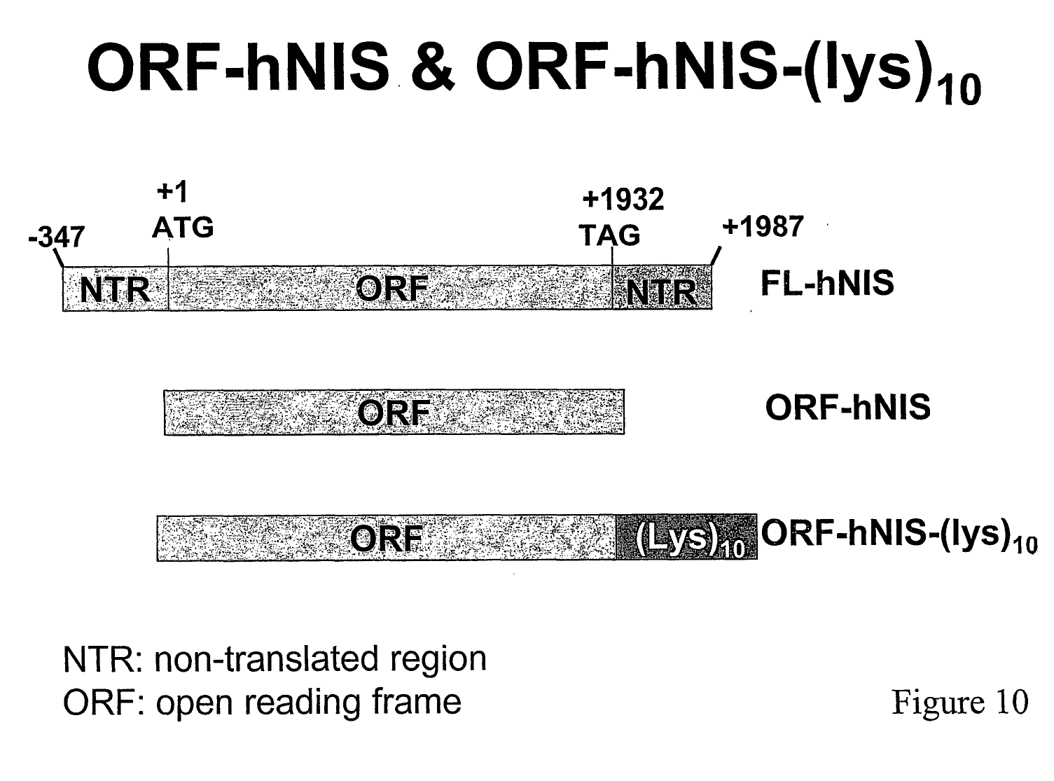

The wild-type hNIS polynucleotide sequence (FIG. 1) was modified to insert a polynucleotide sequence at the 3′ end of the translated region of the NIS encoding region that encoded 10 lysine amino acids. Such a polynucleotide encoding a modified NIS protein, called ORF-hNIS-(lys)₁₀, encoded a modified hNIS protein that has a stretch of 10 lysine amino acids at the C-terminus of the protein which are not present in the wild-type protein. FIG. 10 shows representations of hNIS polynucleotide sequences. At the top of FIG. 10 is a cDNA encoding wild-type hNIS (FL-hNIS). The polynucleotide sequence shown also includes both 5′ and 3′ non-translated sequences. The polynucleotide sequence represented in the middle of FIG. 10 is the hNIS open reading frame (ORF) without the non-translated regions (ORF-hNIS). The polynucleotide sequence represented at the bottom of FIG. 10 has the ten lysine amino acids inserted into the 3′ coding region ((ORF-hNIS-(lys)₁₀).

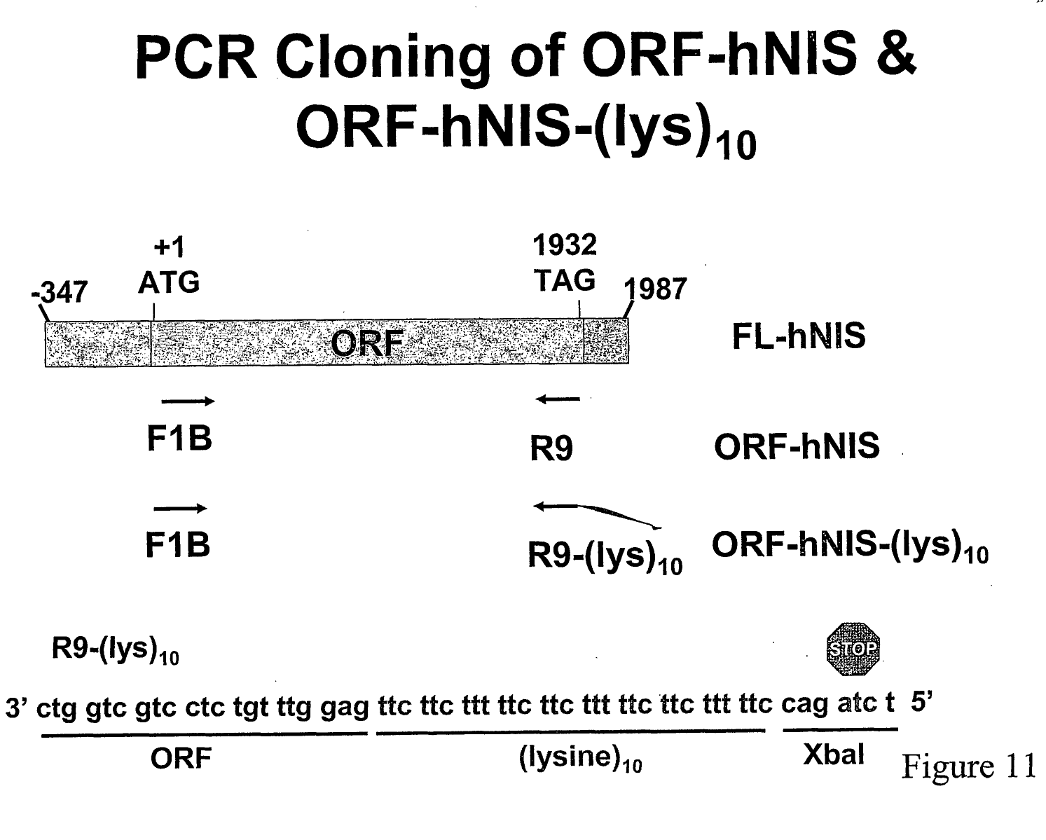

FIG. 11 shows the steps used to obtain the ORF-hNIS-(lys)₁₀ polynucleotide sequence. Two consecutive PCR steps were used. In the first PCR step, the FL-hNIS polynucleotide sequence was used as template in a PCR reaction using two primers. The first primer, F1B, annealed to the 5′ end of the NIS ORF. The second primer, R9, annealed to the 3′ end of the NIS ORF. The product of the PCR reaction using these primers was ORF-hNIS, the NIS polynucleotide sequence in which the non-translated regions of the polynucleotide sequence had been removed. In the second PCR reaction, the ORF-hNIS reaction product of the first PCR reaction was used as the template. Two primers were used in the second PCR reaction. The 5′ primer was F1B, identical to the 5′ primer used in the first PCR reaction. The 3′ primer, called R9-(lys)₁₀, contained the R9 primer plus sequences complementary to 10 consecutive codons that encode lysine, a stop codon and a convenient restriction endonuclease cleavage site usable for cloning the gene after completion of the PCR reaction. The sequence of the R9-(lys)₁₀ PCR primer is shown at the bottom of FIG. 11 (SEQ ID NO. 3).

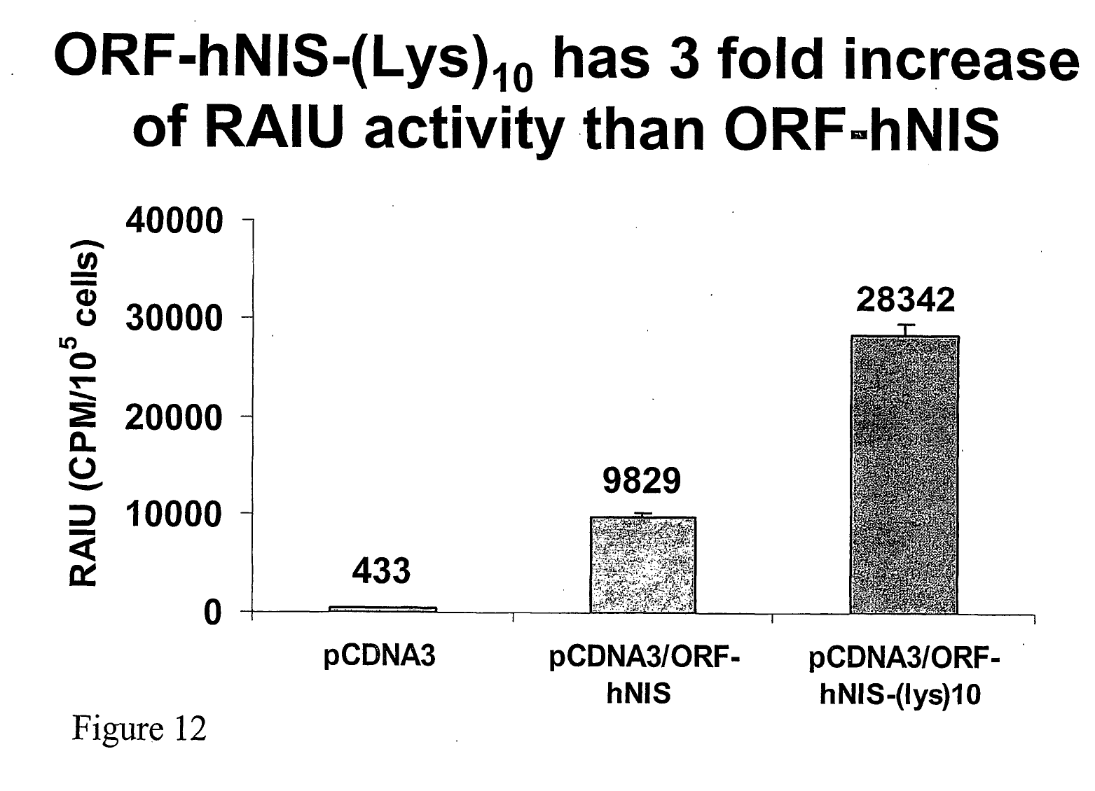

FIG. 12 is a graph showing that the protein encoded by the ORF-hNIS-(lys)₁₀ polynucleotide sequence, encoding the modified NIS protein, resulted in an increased concentration of intracellular radioiodide compared to equivalent expression of wild type NIS protein, ORF-hNIS protein. Cells were transfected with one of three different plasmids: a control, plasmid (pcDNA3); an expression plasmid encoding the wild type NIS protein (pcDNA3/ORF-hNIS); or an expression plasmid encoding the modified hNIS protein with 10 consecutive lysine residues at the N-terminus of the protein (pcDNA3/ORF-hNIS-(lys)₁₀). The cells containing these plasmids and expressing the encoded polynucleotide sequences were assayed for NIS activity using the in vitro RAIU assay, described above in Example 1. The results, shown in FIG. 12, indicated that the insertion of 10 lysines into the C-terminus of NIS molecules resulted in a 3-fold increase of accumulated radioiodide in the cells expressing the gene compared to cells expressing the wild type NIS gene, without the additional lysines.

### REFERENCES

1. Smanik P A et al. Cloning of the human sodium iodide symporter. Biochem Biophys Res Commun 1996; 226: 339-345.
2. Dai G, Levy O, Carrasco N. Cloning and characterization of the thyroid iodide transporter. Nature 1996; 379: 458-460.
5. Cho J Y et al. Expression and activity of human Na⁺/I⁻ symporter in human glioma cells by adenovirus-mediated gene delivery. Gene Ther 2000; 7: 740-749.
10. Goodman J H et al. inhibition of tumor growth in a glioma model treated with boron neutron capture therapy. Neurosurgery 1990; 27: 383-388.
11. Clendenon N R et al. Boron neutron capture therapy of a rat glioma. Neurosurgery 1990; 26: 47-55.
17. Ko L, Koestner A, Wechsler W. Characterization of cell cycle and biological parameters of transplantable glioma cell lines and clones. Acta Neuropathol (Berl) 1980; 51: 107-111.
18. Ko L, Koestner A, Wechsler W. Morphological characterization of nitrosourea-induced glioma cell lines and clones. Acta Neuropathol (Berl) 1980; 51: 23-31.
19. Kobayashi N et al. An improved rat brain-tumor model. J Neurosurg 1980; 53: 808-815.
20. Miller D A, Rosman G J. Improved retroviral vectors for gene transfer and expression. BioTechniques 1989; 980-990.
21. Barth R F et al. Boron neutron capture therapy of brain tumors: enhanced survival and cure following blood-brain barrier disruption and intracarotid injection of sodium borocaptate and boronophenylalanine. Int J Radiat Oncol Biol Phys 2000; 47: 209-218.
22. Jhiang S M et al. An immunohistochemical study of Na⁺/I⁻ symporter in human thyroid tissues and salivary gland tissues. Endocrinology 1998; 139: 4416-4419.
23. Cho J Y et al. Hormonal regulation of radioiodide uptake activity and Na⁺/I⁻ symporter expression in mammary glands. J Clin Endocrinol Metab 2000; 85: 2936-2943.
24. Barth R F et al. Boron neutron capture therapy of brain tumors: enhanced survival following intracarotid injection of either sodium borocaptate or boronophenylalanine with or without blood-brain barrier disruption. Cancer Res 1997; 57: 1129-1136.
25 Levy O et al. Identification of a structural requirement for thyroid Na⁺/I— symporter (NIS) function from analysis of a mutation that causes human congenital hypothyroidism. FEBS Lett 1998; 429: 3640.
26. Eskandari S et al. Thyroid Na+/I symporter. Mechanism, stoichiometry, and specificity. J Biol Chem 1997; 272: 27230-27238.
27. Kotzerke J et al. Pharmacokinetics of ^{99m}Tc-pertechnetate and ¹⁸⁸Re-perrhenate after oral administration of perchlorate: option for subsequent care after the use of liquid ¹⁸⁸Re in a balloon catheter. Nucl Med Commun 1998; 19: 795-801.
28 Lin W Y et al. A comprehensive study on the blockage of thyroid and gastric uptakes of ¹⁸⁸Re-perrhenate in endovascular irradiation using liquid-filled balloon to prevent restenosis. Nucl Med Biol 2000; 27:83-87.
29. Larsen R H, Slade S, Zalutsky M R. Blocking [²¹¹At]astatide accumulation in normal tissues: preliminary evaluation of seven potential compounds. Nucl Med Biol 1998; 25: 351-357.
30. Cobb L M et al. Relative concentration of astatine-211 and iodine-125 by human fetal thyroid and carcinoma of the thyroid in nude mice. Radiother Oncol 1988; 13: 203-209.
31. Castro M R et al. Monoclonal antibodies against the human sodium iodide symporter: utility for immunocytochemistry of thyroid cancer. J Endocrinol. 1999; 163: 495-504.
32. Levy O et al. N-linked glycosylation of the thryroid Na+/I— symporter (NIS). Implications for its secondary structure model. J. Biol. Chem. 273: 22657-22663.

1. A polynucleotide encoding a modified sodium iodide symporter (NIS) protein, the protein having a net electrostatic charge more positive than the net electrostatic charge of a corresponding wild-type NIS protein, wherein expression of the modified NIS protein in a cell results in higher intracellular levels of an NIS substrate than does expression of the same amount of a wild-type NIS protein.

2. The polynucleotide of claim 1, wherein the modified NIS protein comprises from 1 to 19 positively charged amino acids added to the amino acid sequence of a wild-type NIS protein.

3. The polynucleotide of claim 1, wherein the modified NIS protein comprises from 4 to 16 positively charged amino acids added to the amino acid sequence of a wild-type NIS protein.

4. The polynucleotide of claim 1, wherein the modified NIS protein comprises from 6 to 14 positively charged amino acids added to the amino acid sequence of a wild-type NIS protein.

5. The polynucleotide of claim 2, wherein the amino acids added to the amino acid sequence of a wild-type NIS protein comprise at least one continuous sequence of amino acids.

6. The polynucleotide of claim 5, wherein the continuous sequence of amino acids contains only positively charged amino acids.

7. The polynucleotide of claim 2 wherein the amino acids added to the amino acid sequence of a wild-type NIS protein comprise 1, 2 or 3 sequences of continuous amino acids.

8. The polynucleotide of claim 5, wherein the continuous sequence of amino acids comprises at least 1 lysine amino acid.

9. The polynucleotide of claim 5, wherein the continuous sequence of amino acids comprises at least 5 lysine amino acids.

10. The polynucleotide of claim 5, wherein the continuous sequence of amino acids comprises at least 10 lysine amino acids.

11. The polynucleotide of claim 5, wherein the continuous sequence of amino acids is added to the amino terminal end, the carboxyl terminal end, or both the amino terminal and carboxyl terminal ends of a wild-type NIS protein.

12. The polynucleotide of claim 5, wherein the continuous sequence of amino acids is added internal to the carboxyl and amino terminal ends of the wild-type NIS protein.

13. The polynucleotide of claim 12, wherein the continuous sequence of amino acids is added to an extra-membrane domain of the protein.

14. The polynucleotide of claim 13, wherein the extra-membrane domain is an intracellular extra-membrane domain.

15. The polynucleotide of claim 1, wherein the modified NIS protein comprises at least one positively charged amino acid that has replaced at least one uncharged or negatively charged amino acid within the amino acid sequence of a wild-type NIS protein.

16. The polynucleotide of claim 1, wherein the modified NIS protein comprises from 1 to 19 positively charged amino acids that have replaced uncharged or negatively charged amino acids within the amino acid sequence of a wild-type NIS protein.

17. The polynucleotide of claim 1, wherein the modified NIS protein comprises from 4 to 16 positively charged amino acids that have replaced uncharged or negatively charged amino acids within the amino acid sequence of a wild-type NIS protein.

18. The polynucleotide of claim 1, wherein the modified NIS protein comprises from 6 to 14 positively charged amino acids that have replaced uncharged or negatively charged amino acids within the amino acid sequence of a wild-type NIS protein.

19. The polynucleotide of claim 15, wherein the uncharged or negatively charged amino acid is within an intracellular extra-membrane domain of the wild-type NIS protein.

20. The polynucleotide of claim 19, wherein the extra-membrane domain is an intracellular extra-membrane domain.

21. The polynucleotide of claim 1, wherein the modified NIS protein comprises at least one uncharged amino acid that has replaced at least one negatively charged amino acid within the amino acid sequence of a wild-type NIS protein.

22. The polynucleotide of claim 1, wherein the modified NIS protein comprises one or more additions of positively charged amino acids to the wild-type NIS protein sequence, replacement of one or more amino acids of the wild-type NIS protein sequence, or combinations of additions and replacements.

23. An expression vector comprising the polynucleotide sequence of claim 1.

24. A modified sodium iodide symporter (NIS) protein having a net electrostatic charge more positive than the net electrostatic charge of a wild-type NIS protein, wherein expression of the modified NIS protein in a cell results in higher intracellular levels of an NIS substrate than does expression of the same amount of a wild-type NIS protein.

25. A method for increasing the intracellular concentration of one or more NIS substrates in a cell, comprising:
a) introducing a modified NIS protein into the cell; and
b) contacting the cell with one or more NIS substrates;
wherein the NIS substrates are transported into the cell.

26. The method of claim 25, wherein the modified NIS protein is introduced into the cell by an expression vector comprising a polynucleotide encoding a modified NIS protein, and wherein the vector expresses the modified NIS protein in the cell.

27. A method for imaging cells or tissues in an individual, comprising the steps of:
a) introducing a modified NIS protein into cells in the individual;
b) administering an NIS substrate to the individual such that the NIS substrate contacts and is transported into cells containing the modified NIS protein; and
c) imaging the cells that have transported the NIS substrate.

28. The method of claim 27, wherein the modified NIS protein is introduced into the cells by an expression vector comprising a polynucleotide encoding a modified NIS protein, and wherein the vector expresses the modified NIS protein in the cell.

29. A method for treating cancer in an individual, comprising the steps of:
a) introducing a modified NIS protein into cancer cells in the individual; and
b) administering an NIS substrate to the individual such that the NIS substrate contacts and is transported into cancer cells containing the modified NIS protein;
wherein the cancer cells that have transported the NIS substrate have decreased viability or decreased growth rate as compared to cancer cells that have not transported the NIS substrate.

30. The method of claim 29, wherein the NIS substrate contains a radioactive isotope.

31. The method of claim 29, wherein the NIS substrate has cytotoxic activity.

32. The method of claim 29, wherein the modified NIS protein is introduced into the cancer cells by an expression vector comprising a polynucleotide encoding a modified NIS protein, and wherein the vector expresses the modified NIS protein in the cell.

33. A method for identifying cells in an individual that express a therapeutic protein encoded by an exogenous polynucleotide, comprising:
a) introducing an expression vector comprising a polynucleotide encoding a modified NIS protein and a polynucleotide encoding a therapeutic protein into cells in the individual;
b) expressing the therapeutic protein and the modified NIS protein in the cells into which the vector has been introduced;
c) administering an NIS substrate to the individual such that the NIS substrate contacts and is transported into the cells which express the therapeutic protein and the modified NIS protein; and
d) imaging the cells to determine which cells have transported the NIS substrate;
wherein the cells that have transported the NIS substrate are also cells that contain the therapeutic protein encoded by the exogenous polynucleotide.
